(19) United States
(12) Patent Application Publication
(10) Pub. No.: US 2024/0269378 A1
(43) Pub. Date: Aug. 15, 2024

(54) MEDICATION DELIVERY SYSTEM, PATCH PUMP AND MEDICAMENT DELIVERY DEVICE

(71) Applicant: Becton, Dickinson and Company, Franklin Lakes, NJ (US)

(72) Inventors: Eric SUN, Waltham, MA (US); Richard J. GYORY, Sudbury, MA (US); Mark WOOD, Sterling, MA (US); Dana COTE, Boxford, MA (US); Tieying JIANG, Shrewsbury, MA (US); Alessandro PIZZOCHERO, Chelmsford, MA (US); Randy MOSHER, Methuen, MA (US)

(73) Assignee: Becton, Dickinson and Company, Franklin Lakes, NJ (US)

(21) Appl. No.: 18/567,616

(22) PCT Filed: Jun. 7, 2022
(86) PCT No.: PCT/US2022/032509

§ 371 Date: Dec. 6, 2023

Related U.S. Application Data
(60) Provisional application No. 63/209,344, filed on Jun. 10, 2021.

(51) Int. Cl. A61M 5/145; A61M 5/142; A61M 39/22

(52) U.S. Cl. CPC A61M 5/14566; A61M 5/14248; A61M 39/22; A61M 2205/3331; A61M 2205/3368

(57) ABSTRACT

Disclosed is a medication delivery system comprising: a container for a medium: a plunger disposed in the container; and a lead screw axially fixed with respect to the container and in threaded engagement with the plunger, where the lead screw is disposed inside of the container, and the rotation of the lead screw causes axial displacement of the plunger with respect to the container. A patch pump comprising the medication delivery system and a medicament delivery device comprising the medication delivery system are also disclosed.

### CROSS-REFERENCE TO RELATED APPLICATIONS

This application claim priority under 35 USC § 119(e) from U.S. Provisional Patent Applications No. 63/209,344 filed Jun. 10, 2021, the content of which (including all attachments filed therewith) is hereby incorporated by reference in its entirety

### FIELD OF DISCLOSURE

Generally, exemplary embodiments of the present disclosure relate to the fields of medication delivery devices. More specifically, exemplary embodiments of the present disclosure relate to medication delivery devices where a stopper or plunger is advanced through a reservoir to dispense medication from the reservoir.

### BACKGROUND

Medication delivery devices of the present disclosure can be useful in the field of insulin therapy, for example for the treatment of type 1 diabetes. One method of insulin therapy includes syringes and insulin pens that require a needle stick at each injection, typically three to four times per day that are simple to use and relatively low in cost. Another widely adopted and effective method of treatment for managing diabetes is the use of an insulin pump. Insulin pumps can help the user keep blood glucose levels within target ranges based on individual needs, by continuous infusion of insulin.

In the example of medical applications where medication delivery devices of the present disclosure can be particularly useful is patch pumps. A patch pump is an integrated device that facilitates infusion therapy for diabetic patients. A patch pump combines most or all of the fluidic components, including the fluid reservoir, pumping mechanism and mechanism for automatically inserting the cannula, in a single housing which is adhesively attached to an infusion site on the patient's skin, and does not require the use of a separate infusion or tubing set. A patch pump containing insulin adheres to the skin and delivers the insulin over a period of time via an integrated subcutaneous cannula. Some patch pumps may be configured to include wireless communication with a separate controller device, while others are completely self-contained. Such devices are replaced on a frequent basis, such as every three days, particularly when the insulin reservoir is exhausted.

As patch pumps are designed to be a self-contained unit that is worn by the diabetic patient, it is preferable to be as small as possible so that it does not interfere with the activities of the user. Thus, in order to minimize discomfort to the user, it would be preferable to minimize the overall size of the patch pump. Conventional patch pumps or a syringe-type devices typically include a driving mechanism with a single advancing lead screw inside medium or fluid reservoir or chamber to push, advance, or otherwise apply force on the plunger in order to dispense the medium or fluid out of the chamber. In order to minimize the size of the patch pump, its constituent parts, such as driving mechanisms, should be reduced as much as possible without compromising the accuracy and reliability of device or its feature set. Another desirable feature of patch pumps is accurate fluid measurement.

### SUMMARY

Exemplary embodiments of the disclosure may address at least the above problems and/or disadvantages and other disadvantages not described above. Also, exemplary embodiments are not required to overcome the disadvantages described above, and may not overcome any of the problems described above.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

As would be readily appreciated by skilled artisans in the relevant art, while descriptive terms such as “medium”, “medicament”, “stopper”, “plunger”, “thread”, “syringe”, “motor”, “bridge”, “nut”, “blade”, “cutter”, “slice”, “sliceable”, “gear”, “sharp”, “wall”, “top”, “side”, “bottom,” “upper,” “lower,” “proximal”, “distal”, “container”, “reservoir”, “chamber” and others are used throughout this specification to facilitate understanding, it is not intended to limit any components that can be used in combinations or individually to implement various aspects of the embodiments of the present disclosure.

Exemplary embodiments of the present disclosure provide system components that can facilitate a reduction in the overall size or footprint of a drug delivery device, such as a patch pump, by a configuration of a container, reservoir or barrel for medium or fluid and a mechanism or driving components for advancing a plunger to dispense the medium or fluid from the reservoir or barrel, where the mechanism or driving components can be disposed such that the overall length of the driving components can be reduced compared to conventional designs.

Exemplary implementations of embodiments of the present disclosure provide various feature and component which may be deployed individually or in various combinations.

According to exemplary embodiments of the present disclosure, a system includes a syringe-style drug container, reservoir, or barrel containing a medium or fluid which can be dispensed by pumping device or mechanism configured to advance a plunger disposed inside the barrel on a lead screw, which is also disposed inside, and axially fixed with respect to, the barrel such that the plunger can be advanced by rotation of the lead screw to fill the barrel with a medium or fluid, or to dispense the medium or fluid out of the barrel.

According to another exemplary embodiment of the present disclosure, a pumping device based on a syringe barrel body is provided, for example for use in a pump, where a plunger can be advanced axially with respect to a barrel by a linkage mechanism driven by, for example, a motor through a gearing as appropriate for a desired application to fill the barrel with a medium or fluid, or to dispense the medium or fluid out of the barrel.

According to yet another exemplary embodiment of the present disclosure a collapsible drive mechanism is provided, which can be deployed for example in a pump and uses yet another linkage mechanism comprising for example, pivotally coupled sets of linkages removably coupled to a plunger and attached at opposite ends of a drive shaft.

According to exemplary embodiments of the present disclosure, significant space savings can be achieved by utilizing exemplary implementations of a mechanical drive mechanism including for example lead screw configurations that can reside essentially inside of the syringe barrel as provided in the exemplary embodiments of the present disclosure.

According to still further exemplary embodiments of the present disclosure, a pressure based insulin volume sensor is provided, which can be deployed for example as an insulin cartridge attachment, where the insulin will be drawn from the cartridge rather than being pushed out from the back, for measuring. In exemplary implementations, such insulin cartridge measurement attachment can be advantageously placed within or attached to an insulin delivery device having a positive displacement pumping mechanism that can draw a vacuum.

Various exemplary implementations of exemplary disclosed embodiments where unique combinations of features can be deployed in difference variations include following.

An exemplary variation of a system comprises: a container for a medium; a plunger disposed in the container; and a lead screw axially fixed with respect to the container and in threaded engagement with the plunger, where the lead screw is disposed inside of the container, and the rotation of the lead screw causes axial displacement of the plunger with respect to the container. An exemplary variation of the system further comprises a motor coupled to the lead screw and disposed outside of the container, the motor selectively rotating the lead screw in one of a first rotational direction and a second rotational direction opposite the first rotational direction. Another exemplary variation of the system is wherein the rotating of the lead screw in the first rotational direction advances the plunger distally to inject the medium from the container, and the rotating of the lead screw in the second rotational direction advances the plunger proximally to draw in the medium into the container.

Yet another exemplary variation of a system further comprises a gear mechanism transferring rotation of the motor to the lead screw.

Yet another exemplary variation of a system is wherein the plunger comprises: a stopper including internal threads engaging external threads of the lead screw to seal off the medium from leaking past the plunger; and a driver including internal thread engaging the external thread of the lead screw to advance the plunger when the lead screw is rotating, wherein at least one of the driver and the stopper comprises an exterior surface to seal to an inside diameter of the container.

Another exemplary variation of a system is wherein the plunger is unitarily formed to comprise the stopper, the driver, and the exterior surface.

An exemplary variation of a system comprises: a container for a medium; a plunger disposed in the container; and a linkage pivotally connected to the plunger for advancing the plunger distally to dispense the medium from said container, the linkage comprising a pivot, a first arm, and a second arm pivotally connected to the first arm at the pivot, wherein pivotal movement of the first arm with respect to the second arm at the pivot causes axial displacement of the plunger with respect to the container. Another exemplary variation of the system further comprises a motor coupled to the linkage and disposed outside of the container, the motor selectively causing the pivotal movement of the first arm with respect to the second arm thereby selectively changing an angle between the first arm and the second arm at the pivot. Another exemplary variation of the system is wherein the pivotal movement increasing the angle between the first arm and the second arm advances the plunger distally to inject the medium from the container, and the pivotal movement decreasing the angle between the first arm and the second arm advances the plunger proximally to draw in the medium into the container.

Another exemplary variation of the system further comprises a gear mechanism operatively coupling the motor to the linkage.

Another exemplary variation of the system is wherein a distal end of the first arm is pivotally connected to the plunger, a distal end of the second arm is pivotally connected to a proximal end of the first arm, and a proximal end of the second arm is connected to the gear mechanism.

Another exemplary variation of the system is wherein the gear mechanism is axially fixed with respect to the container.

Another exemplary variation of the system is wherein the plunger comprises: a stopper including an exterior surface to seal to an inside diameter of the container; and a driver including the pivotal connection to the linkage.

Another exemplary variation of the system is wherein the plunger is unitarily formed to comprise the stopper and the driver.

An exemplary variation of a system comprises: a container for a medium; a plunger disposed in the container; a linkage mechanism connected to the plunger for advancing the plunger distally to dispense the medium from said container, the linkage mechanism comprising a first linkages, a second linkage, a third linkage, a fourth linkage, a first pivot, a second pivot, a third pivot, and a fourth pivot; and a drive shaft disposed at a proximal end of the container and connected to the linkage mechanism, wherein a distal end of the first linkage and a distal end of the second linkage are pivotally coupled at the first pivot and connected to the plunger, a proximal ends of the first linkage is pivotally coupled to a distal ends of the third linkage at the second pivot, a proximal ends of the second linkage is pivotally coupled to a distal ends of the fourth linkage at the third pivot, the third linkage and the fourth linkage are pivotally coupled at the fourth pivot, the fourth pivot configured between a proximal end and the distal end of the third linkage and between a proximal end and the distal end of the fourth linkage, the proximal end of the third linkage is connected to the drive shaft at a first connection, and the proximal end of the fourth linkage is connected to the drive shaft at a second connection, and wherein axial displacement of the first connection with respect to the second connection causes axial displacement of the plunger with respect to the container.

Another exemplary variation of the system further comprises a motor coupled to drive shaft, the motor selectively causing rotational movement of the drive shaft resulting in the axial displacement of the first connection with respect to the second connection.

Another exemplary variation of the system comprises is wherein decreasing the axial displacement of the first connection with respect to the second connection advances the plunger distally to inject the medium from the container, and increasing the axial displacement of the first connection with respect to the second connection advances the plunger proximally.

Another exemplary variation of the system further comprises a gear mechanism operatively coupling the motor to the drive shaft.

Another exemplary variation of the system is wherein the axial displacement of the first connection with respect to the second increases due to rotation of the dive shaft in a first rotational direction, and decreased due to rotation of the dive shaft in a second rotational direction opposite to the first rotational direction.

Another exemplary variation of the system is wherein the plunger comprises: a stopper including an exterior surface to seal to an inside diameter of the container; and a driver including the connection to the linkage mechanism.

Another exemplary variation of the system is wherein the plunger is unitarily formed to comprise the stopper and the driver.

Exemplary variations of the systems include those wherein the container comprises an endcap disposed at a distal end portion of the container, the endcap comprising at least one of an outlet for dispensing the medium and an inlet for filling the container.

An exemplary variation of a system comprises: a pressure release valve; and a sensor component including at least one of a pressure sensor and a temperature sensor, wherein the pressure release valve and the sensor component are configured with respect to a back end of a cartridge, the cartridge comprising a plunger and insulin to be drawn from the cartridge, and when the insulin is drawn from the cartridge and the plunger moves by an amount of movement toward a front end of the cartridge, thereby increasing the volume between the plunger and the back end of the cartridge, and when the pressure-release valve is set off by the increase of the volume, the sensor component measures a transient response of reaching equilibrium, after the pressure-release valve is set off based on output of at least one of the pressure sensor and the temperature sensor, to derive the amount of movement by the plunger.

A patch pump can comprise any of exemplary variations of the disclosed systems. Also any medicament delivery device can comprise any of exemplary variations of the disclosed systems.

### BRIEF DESCRIPTION OF THE DRAWINGS

The above and/or other example aspects and advantages will become apparent and more readily appreciated from the following description of example embodiments, taken in conjunction with the accompanying drawings in which:

FIGS. 1A and 1B are examples of perspective views of an exterior of a device according to exemplary embodiments of the present disclosure.

FIGS. 2A, 2C, and 2B diagrammatically illustrates a combination of system components according to an exemplary embodiment of the present disclosure.

FIG. 3 illustrates an examples of a cross-sectional view of a plunger assembly of a device according to exemplary embodiments of the present disclosure.

FIG. 4 illustrates diagrammatic and cross-sectional views of conventional device components.

FIGS. 5 and 6 illustrate detailed view of portions of a cross-sectional view of an exemplary plunger assembly as shown in FIG. 3.

FIG. 7 illustrates an example of a perspective view of components of a device according to another exemplary implementation of exemplary embodiment of the disclosure.

FIG. 8 diagrammatically shows a top view of components of a device according to another exemplary implementation of exemplary embodiment of the disclosure.

FIG. 9 illustrates an example of a perspective view of a pumping device and components according to another exemplary embodiment of the disclosure.

FIG. 10 is a perspective cut-out view of a portion of a pumping device and components according to exemplary implementation of the another exemplary embodiment of the disclosure illustrated in FIG. 9.

FIGS. 11A, 11B, and 11C diagrammatically illustrate operation and components of device according to exemplary implementation of the another exemplary embodiment of the disclosure illustrated in FIG. 9.

FIG. 12 illustrates an example of a perspective view of a drive mechanism and components according to yet another exemplary embodiment of the disclosure.

FIGS. 13 and 14 diagrammatically illustrate operation and components of device according to exemplary implementation of the yet another exemplary embodiment of the disclosure illustrated in FIG. 12.

FIG. 15 is a perspective cut-out view of a portion of a drive mechanism and components according to exemplary implementation of the yet another exemplary embodiment of the disclosure illustrated in FIG. 12.

FIG. 16 is block diagram of an exemplary system a pressure based insulin volume sensor according to still another exemplary embodiment of the disclosure.

FIG. 17 is a circuit-type block diagram of an exemplary implementation of various components of a pressure based insulin volume sensor according to the still another exemplary embodiment of the disclosure illustrated in FIG. 16.

### DETAILED DESCRIPTION OF EXEMPLARY EMBODIMENTS

Referring now to the drawings, wherein like reference numerals designate identical or corresponding parts throughout the several views, embodiments of the present disclosure are described as follows.

It will be understood that the terms “include,” “including,” “comprise,” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,” “-er (-or),” and “module” described in the specification refer to an element for performing at least one function or operation, and may be implemented in hardware, software, or the combination of hardware and software.

Various terms are used to refer to particular system components. Different companies may refer to a component by different names—this document does not intend to distinguish between components that differ in name but not function.

Matters of these exemplary embodiments that are obvious to those of ordinary skill in the technical field to which these exemplary embodiments pertain may not be described here in detail. In addition, various features of the exemplary embodiments can be implemented individually or in any combination or combinations, and would be understood by one of ordinary skill in the art of medicament delivery devices.

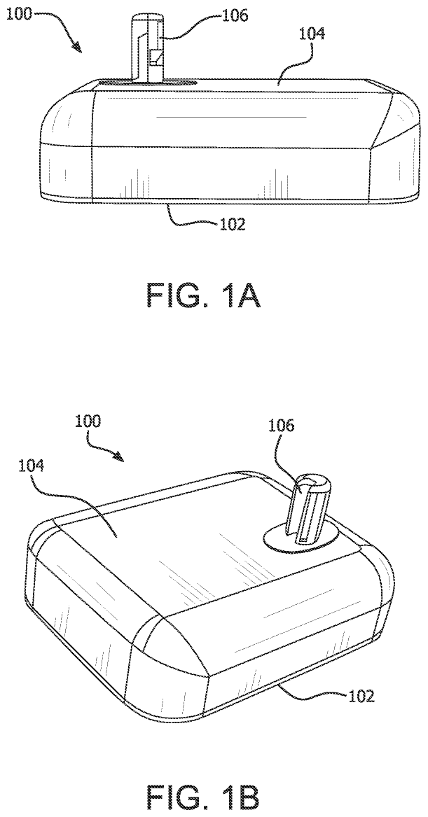

Exemplary embodiments of the present disclosure can be applied to a pump concept, such as for example a wearable disposable patch pump 100 configured to include a base 102, outer housing 104, and an insertion mechanism 106, as shown in perspective views of FIGS. 1A and 1B.

### Lead Screw Infusion Pump

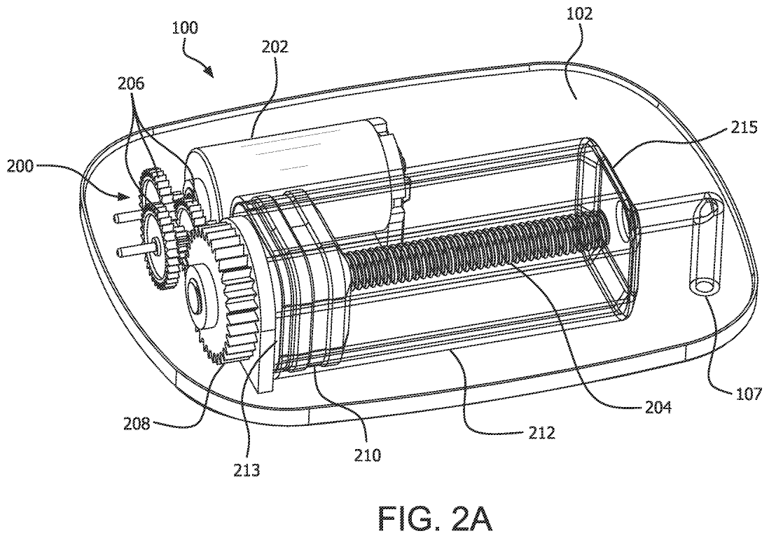

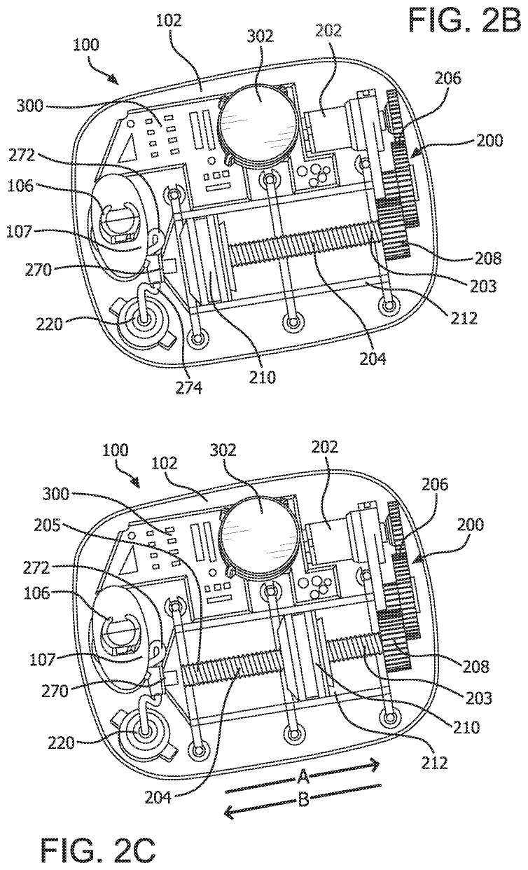

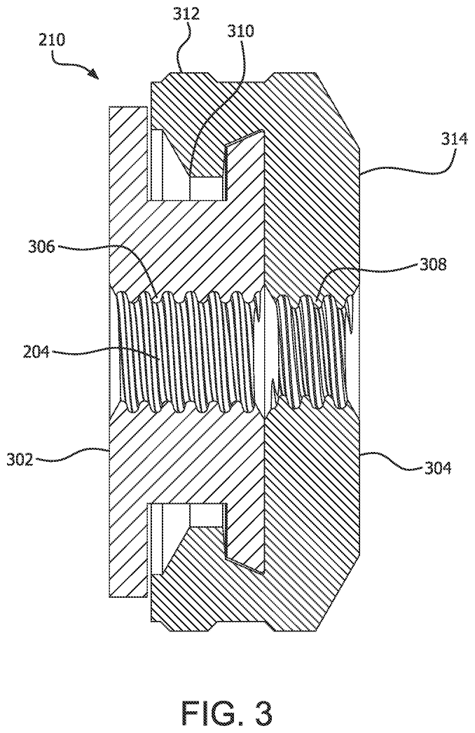

FIG. 2A is an example of a perspective view, and FIGS. 2B and 2C are an example of a more detailed top view, of pump 100 without the outer housing or cover 104, and diagrammatically shows at least some of the various components that can be configured on base 102 of a pump 100 according to an exemplary implementation of the embodiments of the preset disclosure. In an exemplary implementation, lead screw infusion pump 100 includes a pumping mechanism 200 including for example a motor 202 configured to rotate lead screw 204. In an exemplary implementation, motor 202 is operatively connected to lead screw 204 by, for example one or more gears including for example and without limitation reduction gears 206 and lead screw gear 208. Motor 202 can be connected to a power source such as a battery 302 and controlled by electronics (which may include programmable microprocessors, memory modules, and wire and/or wireless communication modules) disposed on PCB 300. Plunger assembly 210 is configured in a syringe-style drug container or barrel 212 to dispense medium or fluid from barrel 212 via an outlet 107 in fluid communication with barrel 212. Examples of configurations of a pumping mechanism 200 and syringe-style drug container or barrel 212 with leadscrew 204 and plunger assembly 210 disposed inside the barrel 212, according to exemplary implementations of the present disclosure, are described in more detail below.

According to exemplary embodiments of the disclosure, lead screw infusion pump 100 includes a lead screw 204 disposed inside barrel 212 such that lead screw 204 extends between proximal end 213 and distal end 215 of barrel 212 and is axially fixed with respect to barrel 212. Plunger assembly 210 is disposed inside barrel 212 on lead screw 204, and plunger assembly 210 is rotationally fixed with respect to barrel 212 and is in threaded engagement with lead screw 204, where lead screw 204 is threaded through plunger assembly 210 such that plunger 210 translates or moves axially with respect to barrel 212 and along lead screw 204 due to rotation of lead screw 204 with respect to barrel 212. Pump 100 is intended to deliver fluids, such as insulin or other hormones, antibiotics, chemotherapy drugs, or pain relievers, contained in barrel 212 into a patient's body via outlet 107 and insertion mechanism 106.

In an exemplary implementation, pump 100 can be initially filled by reversing the motor 202. The plunger 210 can start at distal end 205 of the lead screw 204 with an empty barrel 212, as illustrated in the example of FIG. 2B. As the motor 202 rotates in reverse, the gear driven lead screw 204 rotates forcing the plunger 210, which is in threaded communication with lead screw 204, to move axially with respect to barrel 212 toward proximal end 203 of lead screw 204, thereby pulling fluid through the inlet 220 filling the barrel 212, as illustrated by arrow A in FIG. 2C, until for example where the plunger is in most proximal position as illustrated in FIG. 2A. Fluid can be dispensed by rotating the motor 202 forward driving the plunger 210 to move axially with respect to barrel 212 and distally into the fluid filled syringe barrel 212, as illustrated by arrow B in FIG. 2C, forcing fluid out of the barrel outlet 107.

FIGS. 3, 5 and 6 diagrammatically illustrate general concepts of a lead screw 204 and plunger assembly 210 configuration according to exemplary embodiments of the present disclosure. As diagrammatically shown in the example of FIG. 3, in an exemplary implementation, plunger assembly 210 can comprise two component: a driver 302 having an internal driver thread 306; and a stopper 304 having an internal stopper thread 308. As further illustrated in the example of FIG. 3, internal threads 306 and 308 are coaxial with respect to each other as well as the axis of the lead screw 204, which is threaded through driver 302 and stopper 305 of plunger assembly 210 via respective internal threads 306 and 308.

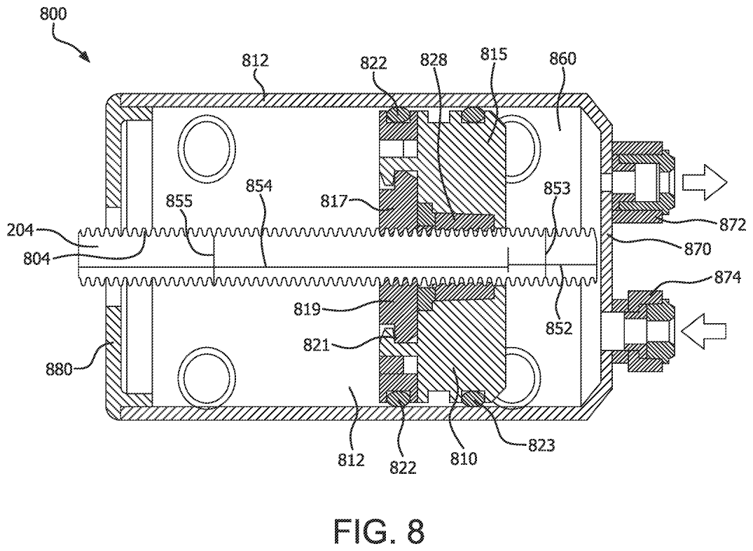

According to an exemplary implementation, driver 302 is, or comprises, a rigid supporting structure for stopper 304. Internal thread 306 of driver 302 can be used to accurately and positively translate plunger 204 with respect to barrel 212 and lead screw 204. In an exemplary implementation driver 302 is axially and rotationally fixed with respect to stopper 304, either permanently or removably, for example by a connection 310/821 (as shown in examples of FIGS. 3 and 8), such as for example and without limitation a snap-fit, frictional, interlock, or other secure connection. In a further exemplary implementations, driver 302 may be unitarily formed from non-metallic and metallic materials, such as polymeric materials, including, but not limited to, thermoplastics, stainless steels or other metallic alloys.

According to an exemplary implementation, stopper 304 is, or comprises, a compliant sealing component. Internal thread 308 of stopper 304 can be used to seal off fluids inside barrel 212 from leaking past the plunger 210. In an exemplary implementation, outer diameter of stopper 304 can comprise a rib or ribs 312 to seal to the inside diameter of the barrel 212, where for example stopper 304 configured such that its surface 314 faces fluid inside barrel 212. In a further exemplary implementations, stopper 304 may be unitarily formed from non-metallic, such as polymeric materials, including, but not limited to, thermoplastics, elastomers, silicones and combinations thereof (for example, copolymers of thermoplastics/elastomers).

According to an exemplary implementation, lead screw 204 is, or comprises, a rigid component driven by a motor 202 directly or through a gear reduction box 206/208. In an exemplary implementation, motor 202 can be controlled by a microprocessor having a memory, such as a microchip mounted on PCB 300, or other controlling method. In a further exemplary implementation, lead screw 204 may be unitarily formed from non-metallic and metallic materials, such as polymeric materials, including, but not limited to, thermoplastics, stainless steels or other metallic alloys. In still further exemplary implementations, a portion of lead screw 204, such as a portion comprising lead screw gear 208, as illustrated in a non-limiting example of FIG. 2A, can be configured outside of barrel 212 in order to operationally connect lead screw 204 to motor 202.

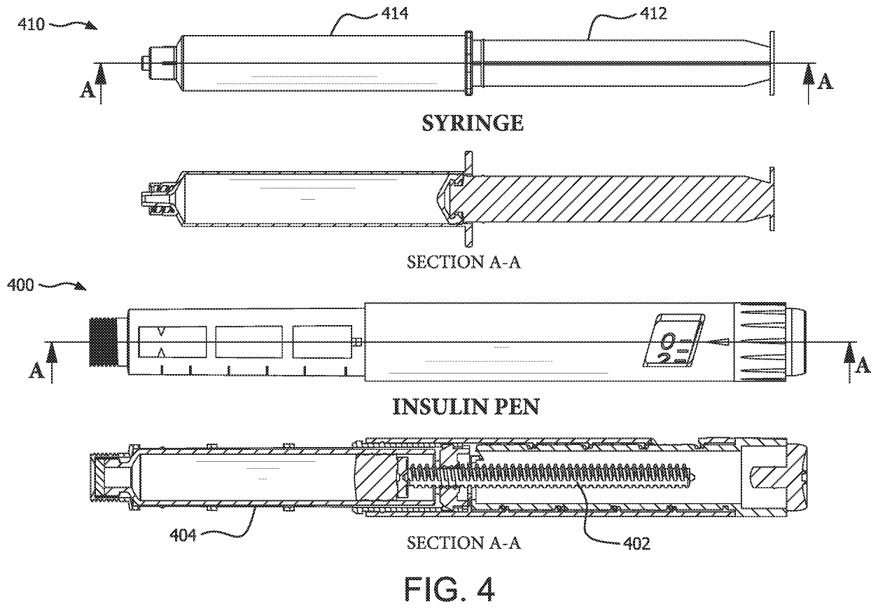

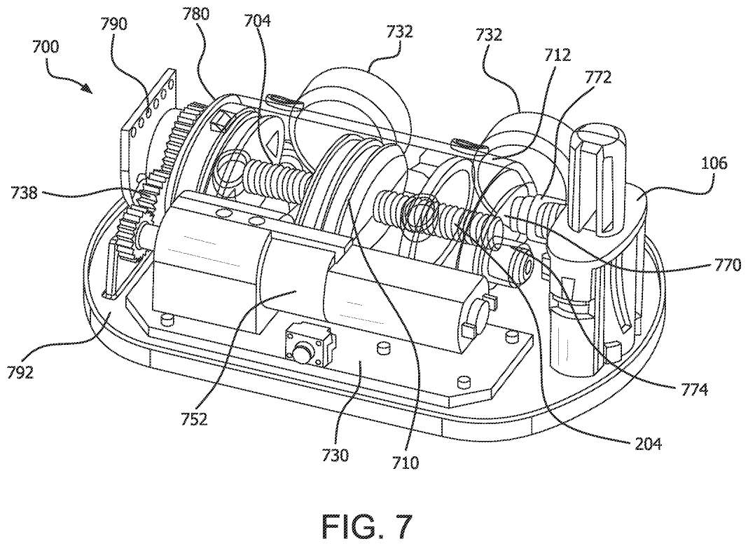

Referring to FIG. 4, which shows a conventional lead screw 402 and plunger rod 412 configurations in a conventional insulin pen 400 and syringe 410, respectively, an exemplary advantage of a lead screw infusion pump, such as pump 100 or 700 according to exemplary embodiments of the disclosure, is a shortened length. A conventional syringe pump or insulin pen is roughly twice the length of its barrel or cartridge 404/414. In such convention systems, syringe plunger rod 412 or insulin pen lead screw 402 resides outside of the fluid reservoir 404/414. The length of syringe plunger rod 412 or insulin pen lead screw 402 outside of fluid reservoir 404/414 needs to be at least as long as the fluid length in the barrel or cartridge 404/414, as illustrated in FIG. 4. In contrast, according to exemplary embodiments of the present disclosure, in lead screw infusion pump 100, lead screw 204 resides inside the barrel 212 at all times, keeping the assemblies length minimally longer than the barrel 212, as shown for example if FIGS. 2A-2C, as well as FIGS. 7 and 8 described in detail below.

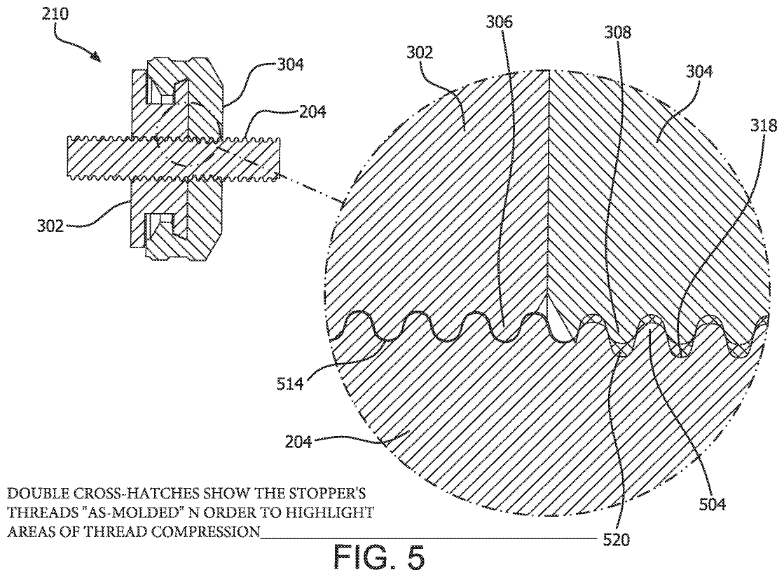

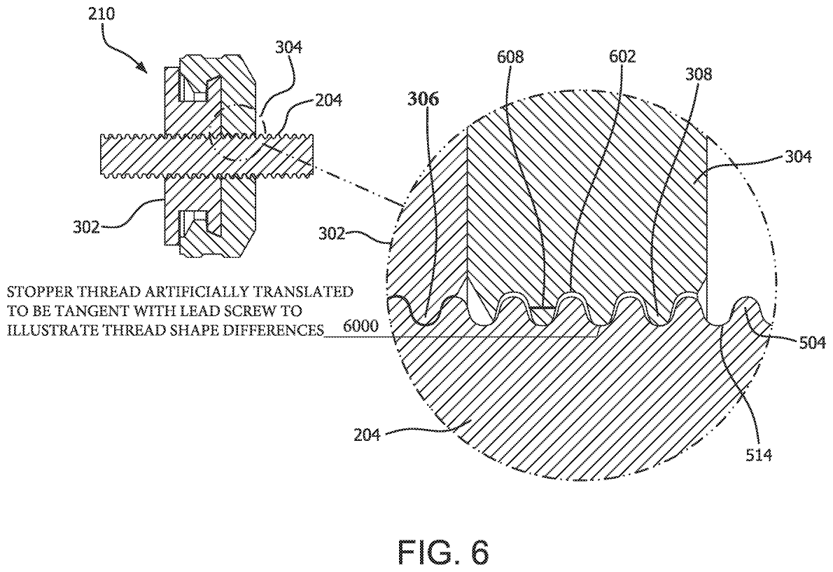

Referring to FIGS. 5 and 6, in exemplary implementations of the embodiments of the present disclosure, where the lead screw 204 is configured inside the barrel 212, threads of plunger assembly 210 seal on the lead screw 204. In exemplary implementations, thread 306 of driver 302 can be used to translate plunger assembly 210 on lead screw 204, while thread 308 of stopper 304 can be designed to compress (denoted by numeral 520 in FIG. 5) and seal on the root 514 of thread 504 of the lead screw 204. Note that, in FIG. 5, the double cross-hatches denoted by numeral 520 show threads 308 of stopper 304 “as molded” in order to highlight areas of thread compression. In an exemplary implementation, minor diameter 318 of thread 308 of stopper 304 can be a percentage smaller than the diameter of root 514 of thread 504 lead screw 204, as illustrated in an example of FIG. 5 (where, without limitation, double cross-hatches show threads 308 of stopper 304 “as molded” in order to highlight areas of thread compression). Further note that, in FIG. 6, as denoted by reference 6000, stopper thread is shown artificially translated to be tangent with lead screw to illustrate thread shape differences. In an exemplary implementation, thread 308 of stopper 304 can be adjusted to thin out in width 608 to allow room 602 for expansion due to the compression, as illustrated in an example of FIG. 6 (where, without limitation, thread 308 of stopper 304 is shown artificially translated to be tangent with lead screw 204 to illustrate thread shape differences).

In exemplary implementations, as illustrated for example in FIGS. 2A, 7 and 8, barrel 212/712/812 and plunger assembly 210/710/810 of lead screw infusion pump 100/700 could have a variety of cross-sections from the common circular to elliptical, oblong, rectangular, or square with rounded corners.

Referring to FIG. 8, in an exemplary implementation of a pumping device 800, which can be deployed for example in a pump 100/700, a stopperless plunger design, such as plunger assembly 810, could be used to seal on the inside diameter of the barrel 212/712/812. For example, an internal compression thread, such as thread 308 illustrated in example of FIGS. 5 and 6, of plunger assembly 710/810 could seal on thread 504/704/804 of lead screw 204. In a another exemplary implementation, an accommodating sub-structure 828 can be configured in plunger assembly 810 around thread 804 of lead screw 204 to allow for compliance, as illustrated in FIG. 8.

In yet another exemplary implementation, thread diameter 853 of the lead screw 204 can be undersized at a length of thread 852 where plunger assembly 210/710/810 is in a parked initial position, such as shown in the example of FIG. 2A, to inhibit creeping of the thermoplastic internal thread of plunger assembly 210/710/810, then increase in diameter 855 for the working length 854 to maintain a compression seal, as illustrated in the example of FIG. 8.

According to an exemplary implementation, a stopperless plunger design, such as a plunger assembly 810, can comprise a rigid outer member 815/817 (including, for example a threaded carrier 819) that includes seals 822/823, such as o-ring seals, that can seal on the inside diameter of barrel 212/712/812 and a compliant elastomer or inner thread 828, such an LSR sleeve, that seals on the lead screw 204, as illustrated in the example of FIG. 8.

Any of the two-piece plunger assembly designs, such as plunger assemblies 210/710/810 could alternatively be manufactured as a single co-molded component. According to exemplary implementations, a compressible compliant plunger assembly can comprise an outer surface and an internal female thread used to seal fluids, such as filled insulin 860, from the pressurized volume of the barrel 212/712/812 during translation of the plunger 210/710/810.

In exemplary embodiments of the disclosure, distal end 215 of barrel 212/712/812 may include an endcap 270/770/870 to facilitate connection of barrel 212/712/812 to insertion mechanism 106, for example via port or tube 272/772/872, to dispense medium or fluid out of barrel 212/712/812. Endcap 270/770/870 can also be configured to facilitate connection of barrel 212/712/812 to fill port or inlet 220/774/874, for example via a tube such as tube 274, to fill barrel 212/712/812 with medium or fluid, as diagrammatically shown in FIGS. 2A-2C, 7 and 8 by displacement of plunger assemblies 210/710/810.

In an exemplary implementation, proximal end of barrel 212/712/812 may include a gear guide 280/780/880. Also, various configurations of components, such as one or more batteries 302/732, PCB 300/730, gears 206/208/738, motor 202/752, and/or encoder 790, on base 102/792 of a pump 100/700 are possible without limitation according to exemplary implementations of the embodiments of the preset disclosure, as illustrated in FIGS. 2A-2C, 7 and 8.

### Dual Linkage Compact Syringe Pump

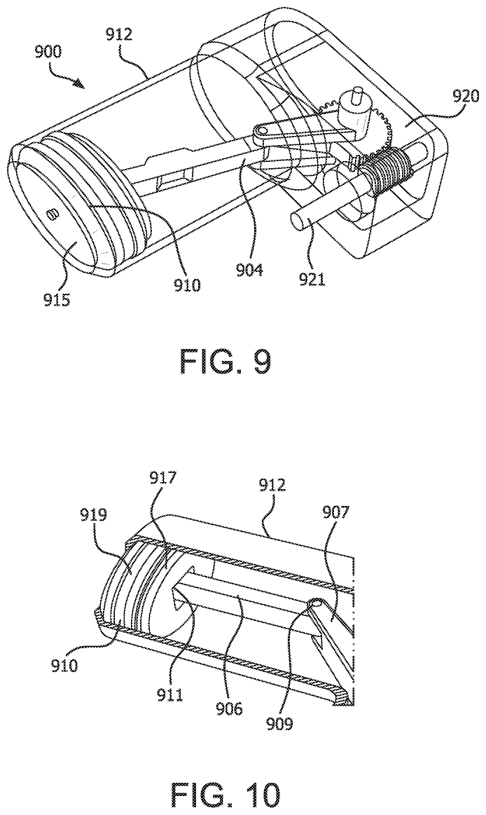

Referring to FIGS. 9, 10 and 11A-11C, another exemplary embodiment of the disclosure provides a pumping device 900 based on a syringe barrel body 912, which can be deployed for example in a pump 100/700. According to the exemplary embodiment, device 900 comprises a plunger 910 disposed in barrel 912, such that plunger 910 can be advanced axially with respect to barrel 912 by a two linkage mechanism 904 driven by, for example a motor/gearbox, such as for example and without limitation motor/gearbox 752 illustrated in FIG. 7, through appropriate gearing 920 (for example operatively connected at 921) comprising for example and without limitation worm or spur gears, as appropriate for a desired application.

According to exemplary implementations, distal end 915 of barrel 912 may comprise an endcap, such as endcap 270/770/870 to facilitate connection of barrel 912 to an insertion mechanism (such as for example insertion mechanism 106), for example via a port or tube (such as for example port or tube 272/772/872), to dispense medium or fluid out of barrel 912. Such endcap of barrel 912 can also be configured to facilitate connection of barrel 912 to fill port or inlet (such as for example fill port or inlet 220/774/874), for example via a tube (such as tube 274), to fill barrel 912 with medium or fluid.

Referring to FIG. 10, which shows a cut-out view barrel 912, according to an exemplary implementation, linkage mechanism 904 comprises: a first arm 906, whose distal end is pivotally connected 911 to plunger 910; and a second arm 907, whose distal end is pivotally connected 909 to proximal end of the first arm 906. Proximal end of the second arm 907 can be connected to gearing 920 as shown in FIG. 9. In an exemplary implementation, plunger 910 can comprise a rigid driver 917 providing pivotal connection 911 to first arm 906, and a stopper 919 providing an outer surface to seal fluids, such as filled insulin 860, from the pressurized volume of the barrel 912 during translation of the plunger 910. As noted with regard to embodiments of FIGS. 2-8, two-piece plunger assembly designs, such as plunger assemblies 210/710/810/190 could alternatively be manufactured as a single co-molded component.

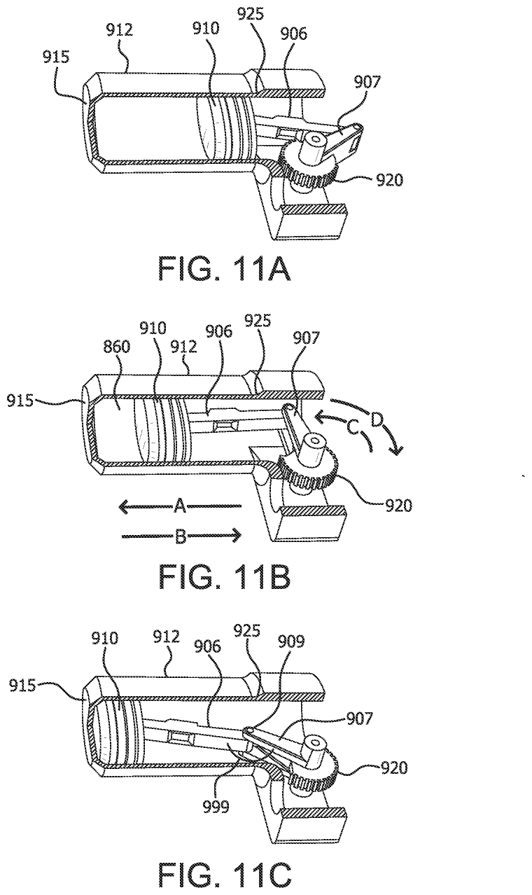

Referring to FIGS. 11A, 11B, and 11C, exemplary operation of a pump comprising pumping device 900 is described as follows. The pump starts, for example, at a rest position with plunger 910 at proximal end 925 of barrel 912, where in such a position, the largest (or longest) portions of linkage mechanism 904 extend out of proximal end of barrel 912. Should barrel 912 be filled with fluid 860 when pump starts at rest position, then when the motor (such as motor 202/742) drives the second arm 907 (input drive linkage) via gearing 920, the input drive linkage 907 moves (C) and drives the first arm 906 (driven linkage) thereby pushing the plunger 910 forward (A) toward distal end 915 of barrel 912 and delivering fluid 860 out of barrel 912.

Using the same mechanism, but in reverse, barrel 912 can be filled at time of use, For exempla, when plunger 910 starts at distal end 915 of barrel 912, then when the motor (such as motor 202/742) drives the second arm 907 (input drive linkage) via gearing 920 in reverse, the input drive linkage 907 moves (D) and drives the first arm 906 (driven linkage) thereby pushing the plunger 910 backward (B) toward proximal end 925 of barrel 912 and drawing fluid 860 into barrel 912.

According to exemplary implementations, encoders (such as encoder 790) may be added to gearbox 920 or alongside the barrel 912 to track the position of stopper or plunger 910 position and provide feedback to the drive electronics, such as those on PCB 300/730. Alternatively, in an exemplary implementation, once the geometry of linkage mechanism is fixed, a kinematic curve may be preprogrammed and motor (gearbox) rotation may be correlated to piston stopper (plunger 910) position.

An exemplary advantage of pumping device 900 is that its configuration does not use a reversing motion over areas previously contacted by insulin 860. Pumping device 900 provides a compact means for driving a single-stroke syringe pump. Further, exemplary implementations of pumping device 900 design may improve noise issues observed in some conventional designs.

An exemplary non-limiting implementation of a pumping device 900 comprise an elliptical cross-section barrel 912 (approximately 10×17 mm cross section) and has a total length from distal end of barrel 912 to initial outer edge of linkage mechanism 904 at a start (rest position) of approximately 43 mm. Without departing from the scope and teaching of the pumping device 900 configuration, it can be appreciated the other linkage geometries or barrel sizes may be used to affect torque, forces, and overall size. Tradeoffs may be considered to fit smaller or larger variants depending on device needs as well as motor/gearbox capability (as well as battery capacity). For example, a design consideration can be to prevent locking or over pivoting of first arm 906 with respect to second arm 907 about pivotal connection 909 by ensuring that the angle 999 remains less than 180 degrees during pump use.

According to exemplary implementations, the design of pumping device 900 can be set-up to balance forces and torque as much as possible while minimizing part count. The design may be further tweaked to change the relationship between input drive angle and output stroke. In an exemplary embodiment, the relationship between input drive angle and output stroke is mostly linear with a non-linear trend at the end of stroke. In an exemplary implementation such design can reduce overall size of pumping device 900. Exemplary implementation of design according to exemplary embodiment are adjustable via equations that characterize the design pivot and linkage lengths. In exemplary implementations, more linear kinematics may be defined and torque may be further affected by changing stopper or plunger 910 geometry, materials, and compression. Backpressure can be accounted for in such analysis.

### Scissor Jack Linkage Drive Mechanism For Syringe Injection System

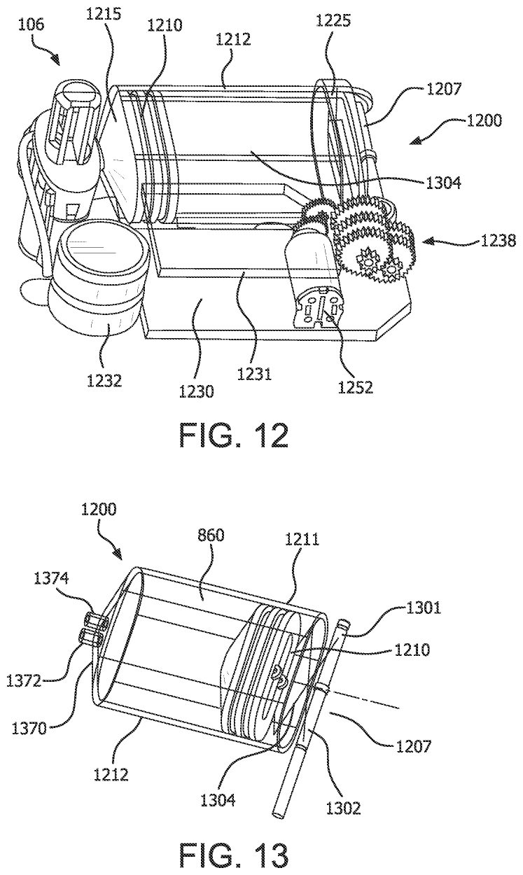

Referring to FIGS. 12-15, yet another exemplary embodiment of the disclosure provides a collapsible drive mechanism 1200, which can be deployed for example in a pump 100 and uses a linkage mechanism 1304 to reduce the overall length of a syringe-based drug-infusion system such as pump 100. According to the exemplary embodiment, a plunger 1210 is disposed in barrel 1212, such that plunger 1210 can be advanced axially with respect to barrel 1212 by mechanism 1200 driven by, for example a motor 1252, such as for example and without limitation motor 202/752 illustrated in FIGS. 2A and 7, through appropriate gearing 1238.

According to exemplary implementations, distal end 1215 of barrel 1212 may comprise an endcap 1370, such as endcap 270/770/870 to facilitate connection of barrel 1212 to an insertion mechanism (such as for example insertion mechanism 106), for example via a port or tube 1372 (such as for example port or tube 272/772/872), to dispense medium or fluid out of barrel 1212. Such endcap 1370 of barrel 1212 can also be configured to facilitate connection of barrel 1212 to fill port or inlet 1374 (such as for example fill port or inlet 220/774/874), for example via a tube (such as tube 274), to fill barrel 1212 with medium or fluid. As further illustrated in the example of FIG. 12, other components that can be deployed along with drive mechanism 1200 in a pump 100/700 include, for example, battery or batteries 1232, such as for example and without limitation batteries 302/732 illustrated in FIGS. 2B and 7, and one or more PCBs 1230/1231, such as for example and without limitation PCBs 300/730 illustrated in FIGS. 2B and 7.

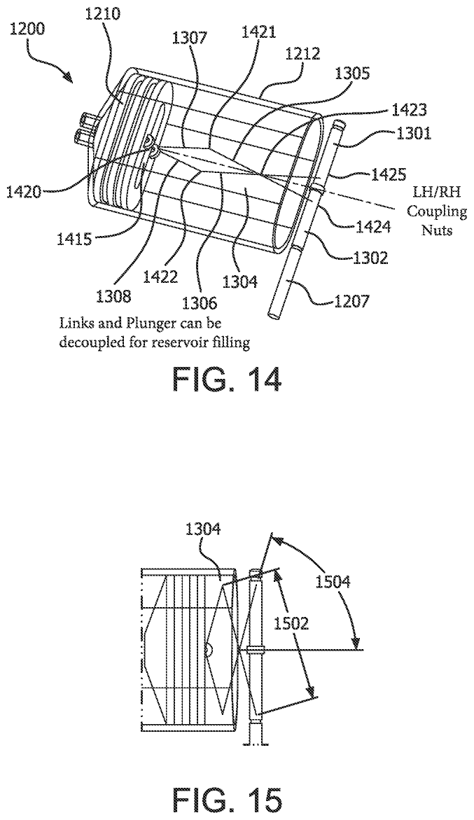

Referring to non-limiting example of FIG. 14 where components according to the exemplary embodiment can be more easily viewed, a drive mechanism can be, or comprises, a collapsible drive mechanism 1200 including a linkage mechanism 1304 comprising, for example, a set of two full linkages 1305 and 1306 and two half linkages 1307 and 1208. In an exemplary implementation, distal ends of half linkages 1307 and 1308 can be joined at, or pivotally coupled to, plunger 1210, for example at proximal end 1415 of plunger 1210, for example via a loose pin 1420. Proximal ends of half linkages 1307 and 1308 can be joined at, or pivotally coupled to, distal ends of respective full linkages 1305 and 1306, for example via respective loose pins 1421 and 1422. Full linkages 1305 and 1306 crisscross and can be joined, or pivotally coupled, essentially at the centers thereof, for example by means of a loose pin 1423. Proximal ends of full linkages 1305 and 1306 are joined at, or pivotally coupled to, the driveshaft 1207 at opposing left 1302 and right 1301 hand female screw threads, respectively, for example by means of respective loose pins 1425 and 1424.

Referring to FIGS. 13 and 14, exemplary operation of a pump comprising drive mechanism 1200 is described as follows. Note that in FIG. 14, links and plunger can be decoupled for reservoir filling. The pump starts, for example, in a retracted position with plunger 1210 at proximal end 1225 of barrel 1212 as illustrated in a non-limiting example of FIG. 14. Should barrel 1212 be filled with fluid 860 when pump starts in a retracted position, then when the motor 1252 (such as motor 202/742) drives the driveshaft 1207 via gearing 1238, the mechanism 1200 is driven by the driveshaft 1207 with opposing left 1302 and right hand 1301 screw threads that can be coupled to the linkage mechanism 1304 via female threads. The driveshaft 1207 when turned will move the linkage mechanism 1304 in a linear direction which pushes the syringe plunger seal 1210 axially with respect to barrel 1212 and injects the drug from barrel 1212 for example and without limitation via an insertion mechanism 106 in fluid communication with barrel 1212.

Accordingly, in an exemplary implementation, when the plunger 1210 is at a filled position, the linkage mechanism 1304 can be retracted and folded up to a compact size, as illustrate in a non-limiting example of FIG. 13. When the driveshaft 1207 is turned by a motor 1252 with gear train 1238, the linkage mechanism 1304 is extended, as illustrated in a non-limiting example of FIG. 14, and the plunger 1210 will eject contents, such as fluid 860 out of barrel 1212 via outlet 1372.

According to exemplary implementation, filling mechanism for a pump comprising a collapsible drive mechanism 1200 can be configured or performed as follows. Plunger seal 1211 (or plunger 1210 at proximal end 1415) can be configured to be decoupled from the linkage mechanism 1304 when plunger 1210 is at starting empty position, such as at a distal end of barrel 1212, as illustrated in FIGS. 12 and 14. This allows filling of barrel 1212 to any desired volume, for example and without limitation up to 3 ml maximum or other defined design limits. In an exemplary implementation, filling can be performed by an external syringe via a fill port or inlet 1374. In exemplary implementations, coupling of plunger 1210 and linkage mechanism 1304 can comprise an active coupling mechanism, such as for example and without limitation a coupling between railroad cars, or a passive coupling mechanism, such as for example and without limitation a push only coupling.

In yet further exemplary implementation, a pinched tube or other mechanism can be configured to temporarily block flow downstream (for example out of outlet 1372) and allow the plunger to expand and/or move toward proximal end of barrel 1212 and hold the drug, such as fluid 869, within barrel 1212. An example of a pinched tube concept can use a downstream tube (such as for example and without limitation tube 272 illustrated in FIGS. 2A and 2B) in a kinked position prior to operation of the catheter injection mechanism 106. After operation of the injection mechanism 106, the tube would become unkinked and allow flow downstream. Potential advantages of such a passive valve, achieved by a pinched tube implementation, can include for example and without limitation reduced complexity, cost, and size compared to alterative valve technology.

An exemplary implementation of a passive downstream flow occlusion mechanism according to an exemplary embodiments can comprise a tube that is manufactured in a kinked position and held in place via a springs of an injection mechanism (such as IM 106). When the injection mechanism is fired, a release collar is moved outward driving the catheter into the skin and freeing the kinked tubing allowing flow downstream.

According to yet other exemplary implementations, temporary downstream occlusion of outlet 1372 can occur by means of a one-way valve, separate pinch valve mechanism or another means in place of the passive kinked tube concept.

According to further exemplary implementations, the number of linkages used in a linkage mechanism 1304, and/or configuration of linkages (such as for example and without limitation linkage length 1502 and jack angle 1504 illustrated in FIG. 15) can be tailored to the desired space savings or other considerations.

According to still further exemplary implementations, which can be potentially applicable to any of disclosed exemplary embodiments: an optical or hall-type sensing mechanism, configured for example on a plunger, can be used to detect movement and fill volume. Alternatively or in combination, a plunger can be used as a visual indicator of full volume through a window on the housing 104. Motor load detection when a drive mechanism couples with the a plunger can be used to detect fill volume.

### Pressure-Based Insulin Volume Sensor For Insulin Cartridge

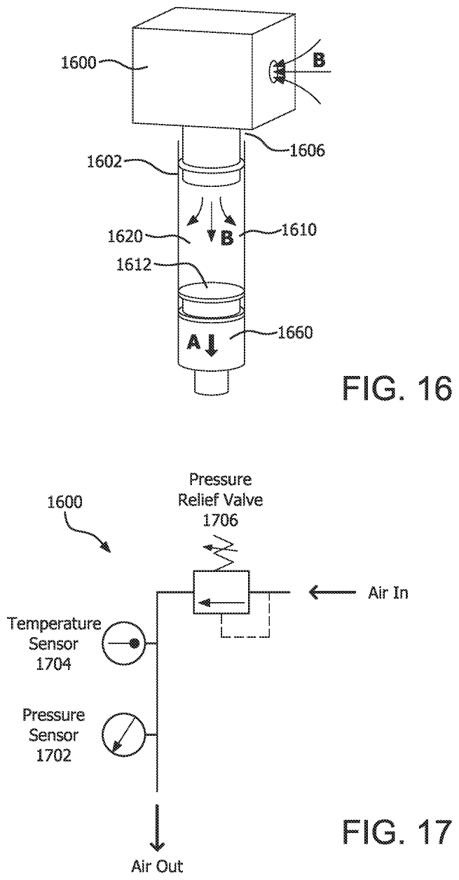

Referring to FIGS. 16 and 17, yet another exemplary embodiment of the disclosure provides a pressure based insulin volume sensor which can be deployed for example as an insulin cartridge attachment 1600 with an on-board pressure release valve 1706 and pressure sensor 1702 and/or temperature sensor 1704 for insulin volume measurement in cartridge through measurement of transient response.

According to exemplary implementations, an attachment 1600 is provided for an insulin cartridge 1602 comprising a plunger 1612, where insulin 1660 will be drawn (A) from the cartridge rather than being pushed out from the back. The attachment 1600 can be connected to the back 1606 of the cartridge 1602 and can use a pressure sensor 1702 and/or temperature sensor 1704 disposed in attachment 1600 to measure the volume of air 1608 in the back region 1610 (behind plunger 1612) of the insulin cartridge 1602. This volume will expand because as insulin is being drawn (A), the plunger 1612 of the syringe will move forward (A) and expand the non-insulin volume 1620 of region 1610 behind the plunger 1612. This increase of volume 1620 will draw a vacuum and then will eventually set off the pressure-release valve 1706. Measuring the transient response (B) of reaching equilibrium will derive how much movement there has been in the plunger 1612 of the insulin cartridge 1602.

Exemplary implementation of the disclosed exemplary embodiment are essentially based on deriving a volume 1620 through the ideal gas law, where by obtaining a temperature and pressure measurement, one can get the volume of the container. For example, there will be a pressure differential that can be measured when the pressure is released, and the time that the pressure takes to reach equilibrium will help derive the movement in the plunger 1612 of the insulin cartridge 1602.

The disclose exemplary embodiment provides an attachment 1600 for an insulin cartridge where the insulin will be drawn from the cartridge rather than being pushed out from the back (such as in exemplary embodiments of FIG. 1A-15). In exemplary implementations, such insulin cartridge measurement can be ideal if placed within an insulin delivery device that has a positive displacement pumping mechanism that can draw a vacuum.

While the present disclosure has been shown and described with reference to certain exemplary embodiments thereof, it will be understood by those skilled in the art that various changes in form and details may be made therein without departing from the spirit and scope of the embodiments of the present disclosure. For example, operative variations and alternative different lead designs may be employed to change dosing resolution, encoders may be used to have feedback of drive mechanism, indexing drives can be employed to repeatably and fail-safe advance the plunger. Generally, for example, non-circular syringe barrel cross-sections may be employed to optimize space utilization and tailor device size to best suit user comfort. Furthermore, any of the features or elements of any exemplary implementations of the embodiments of the present disclosure as describes above and illustrated in the drawing figures can be implemented individually or in any combination(s) as would be readily appreciated by skilled artisans without departing from the spirit and scope of the embodiments of the present disclosure.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of disclosure.

Other objects, advantages and salient features of the disclosure will become apparent to those skilled in the art from the details provided, which, taken in conjunction with the annexed drawing figures, disclose exemplary embodiments of the disclosure.

1. A medication delivery system comprising:
a container for a medium;
a plunger disposed in the container; and
a lead screw axially fixed with respect to the container and in threaded engagement with the plunger,
where the lead screw is disposed inside of the container, and the rotation of the lead screw causes axial displacement of the plunger with respect to the container.

2. The medication delivery system of claim 1, further comprising a motor coupled to the lead screw and disposed outside of the container, the motor selectively rotating the lead screw in one of a first rotational direction and a second rotational direction opposite the first rotational direction.

3. The medication delivery system of claim 2, wherein
the rotating of the lead screw in the first rotational direction advances the plunger distally to inject the medium from the container, and
the rotating of the lead screw in the second rotational direction advances the plunger proximally to draw in the medium into the container.

4. The medication delivery system of claim 2, further comprising a gear mechanism transferring rotation of the motor to the lead screw.

5. The medication delivery system of claim 1, wherein the plunger comprises:
a stopper including internal threads engaging external threads of the lead screw to seal off the medium from leaking past the plunger; and
a driver including internal thread engaging the external thread of the lead screw to advance the plunger when the lead screw is rotating,
wherein at least one of the driver and the stopper comprises an exterior surface to seal to an inside diameter of the container.

6. The medication delivery system of claim 5, wherein the plunger is unitarily formed to comprise the stopper, the driver, and the exterior surface.

7. A medication delivery system comprising:
a container for a medium;
a plunger disposed in the container; and
a linkage pivotally connected to the plunger for advancing the plunger distally to dispense the medium from said container, the linkage comprising a pivot, a first arm, and a second arm pivotally connected to the first arm at the pivot,
wherein pivotal movement of the first arm with respect to the second arm at the pivot causes axial displacement of the plunger with respect to the container.

8. The medication delivery system of claim 7, further comprising a motor coupled to the linkage and disposed outside of the container, the motor selectively causing the pivotal movement of the first arm with respect to the second arm thereby selectively changing an angle between the first arm and the second arm at the pivot.

9. The medication delivery system of claim 7, wherein
the pivotal movement increasing the angle between the first arm and the second arm advances the plunger distally to inject the medium from the container, and
the pivotal movement decreasing the angle between the first arm and the second arm advances the plunger proximally to draw in the medium into the container.

10. The medication delivery system of claim 7, further comprising a gear mechanism operatively coupling the motor to the linkage.

11. The medication delivery system of claim 10, wherein a distal end of the first arm is pivotally connected to the plunger, a distal end of the second arm is pivotally connected to a proximal end of the first arm, and a proximal end of the second arm is connected to the gear mechanism.

12. The medication delivery system of claim 10, wherein the gear mechanism is axially fixed with respect to the container.

13. The medication delivery system of claim 7, wherein the plunger comprises:
a stopper including an exterior surface to seal to an inside diameter of the container; and
a driver including the pivotal connection to the linkage.

14. The medication delivery system of claim 13, wherein the plunger is unitarily formed to comprise the stopper and the driver.

15. A medication delivery system comprising:
a container for a medium;
a plunger disposed in the container;
a linkage mechanism connected to the plunger for advancing the plunger distally to dispense the medium from said container, the linkage mechanism comprising a first linkages, a second linkage, a third linkage, a fourth linkage, a first pivot, a second pivot, a third pivot, and a fourth pivot; and
a drive shaft disposed at a proximal end of the container and connected to the linkage mechanism,
wherein
a distal end of the first linkage and a distal end of the second linkage are pivotally coupled at the first pivot and connected to the plunger,
a proximal ends of the first linkage is pivotally coupled to a distal ends of the third linkage at the second pivot,
a proximal ends of the second linkage is pivotally coupled to a distal ends of the fourth linkage at the third pivot,
the third linkage and the fourth linkage are pivotally coupled at the fourth pivot, the fourth pivot configured between a proximal end and the distal end of the third linkage and between a proximal end and the distal end of the fourth linkage,
the proximal end of the third linkage is connected to the drive shaft at a first connection, and
the proximal end of the fourth linkage is connected to the drive shaft at a second connection, and
wherein axial displacement of the first connection with respect to the second connection causes axial displacement of the plunger with respect to the container.

16. The medication delivery system of claim 15, further comprising a motor coupled to drive shaft, the motor selectively causing rotational movement of the drive shaft resulting in the axial displacement of the first connection with respect to the second connection.

17. The medication delivery system of claim 15, wherein
decreasing the axial displacement of the first connection with respect to the second connection advances the plunger distally to inject the medium from the container, and
increasing the axial displacement of the first connection with respect to the second connection advances the plunger proximally.

18. The medication delivery system of claim 16, further comprising a gear mechanism operatively coupling the motor to the drive shaft.

19. The medication delivery system of claim 15, wherein the axial displacement of the first connection with respect to the second connection increases due to rotation of the dive shaft in a first rotational direction, and decreased due to rotation of the dive shaft in a second rotational direction opposite to the first rotational direction.

20. The medication delivery system of claim 15, wherein the plunger comprises:
a stopper including an exterior surface to seal to an inside diameter of the container; and
a driver including the connection to the linkage mechanism.

21. The medication delivery system of claim 20, wherein the plunger is unitarily formed to comprise the stopper and the driver.

22. The medication delivery system of claim 1, wherein the container comprises an endcap disposed at a distal end portion of the container, the endcap comprising at least one of an outlet for dispensing the medium and an inlet for filling the container.

23. A medication delivery system comprising:
a pressure release valve; and
a sensor component including at least one of a pressure sensor and a temperature sensor,
wherein
the pressure release valve and the sensor component are configured with respect to a back end of a cartridge, the cartridge comprising a plunger and insulin to be drawn from the cartridge, and
when the insulin is drawn from the cartridge and the plunger moves by an amount of movement toward a front end of the cartridge, thereby increasing the volume between the plunger and the back end of the cartridge, and when the pressure-release valve is set off by the increase of the volume, the sensor component measures a transient response of reaching equilibrium, after the pressure-release valve is set off based on output of at least one of the pressure sensor and the temperature sensor, to derive the amount of movement by the plunger.

24. A patch pump comprising the medication delivery system of claim 1.

25. A medicament delivery device comprising the medication delivery system of claim 1.
